(19) United States
(12) Patent Application Publication
(10) Pub. No.: US 2013/0184539 A1
(43) Pub. Date: Jul. 18, 2013

(54) Sensor Arrangement for Detecting Muscle Activity for the Control of Technical Equipment

(71) Applicant: RSL STEEPER GROUP LIMITED, Kent (GB)

(72) Inventors: Klaus BUCHENRIEDER, Riemerling (DE); Stefan HERRMANN, Unterhaching (DE)

(73) Assignee: RSL STEEPER GROUP LIMITED, Kent (GB)

(21) Appl. No.: 13/678,738

(22) Filed: Nov. 16, 2012

(30) Foreign Application Priority Data
Nov. 16, 2011 (DE) 102011118732.8

(51) Int. Cl. A61B 5/0488; A61B 5/00; A61B 5/11; A61B 5/04; A61B 5/1455; A61B 5/145

(52) U.S. Cl. CPC A61B 5/04888; A61B 5/14552; A61B 5/14546; A61B 5/1107; A61B 5/04012; A61B 5/6801
USPC 600/301; 600/324

(57) ABSTRACT

A sensor arrangement for detecting muscle activity for the control of technical equipment. When in use the arrangement covers a region of the skin surface of a user, to provide a signal indicative of muscle activity in a limited region for subsequent processing. The arrangement has at least one double-differential myoelectric sensor, together with at least one near-infrared sensor. These are for simultaneous or time-delayed derivation of (a) myoelectric activity and (b) the value of a parameter of the blood (for example the blood oxygen content or the relative quantity of haemoglobin) respectively, in the muscle, the muscles or the tissue under the arrangement.

The invention relates to a combined sensor system for deriving muscle signals on the skin surface to control, for example, hand or leg prostheses, or as an input for computers or other devices.

### Prior Art with Sources

It is known to arrange myoelectric sensors close to muscles on the skin surface, the sensors detecting the depolarisation signal emitted to all sides and providing it for further processing [LUCA, ALTER, HERR09]. It is furthermore known that the depolarisation signals are overlaid by simultaneously stimulated, innervated muscles. This effect increases with a growing spacing of the derivation electrodes and is more pronounced in myoelectric sensors with single-differential electrodes than in sensors with double-differential electrodes [FARINA, DELSYS].

It is further indicated that near-infrared spectroscopy (NIRS), an active measuring method for the relative measurement of the blood oxygen content with at least one light transmitter and one receiver [ASLIN, CHALM], is also suitable for investigating muscle activity {AKIMA, QUARE, MIURA01 MIURA03]. The combination of a single-differential myoelectric sensor with a simple near-infrared sensor, consisting of a light source and a photo receiver to detect muscle activities is also found in the literature [HERR10A, HERR10B, HERR11A, HERR11 B]. In the same literature, it is also shown how the derived signals of the two sensors can be synchronously combined as a system by a suitable weighting. A detailed section about the weighted combination of two features in one time window was disclosed in [HERR10C].

### Problem in the Prior Art

Using the previously known combinations of a myoelectric and near-infrared sensor it is only possible to detect one region under the sensor, which cannot be varied during the measurement. The overlays of the myoelectric signals of a plurality of muscles are recorded at a recording point and allow the basic distinction between activity and the rest state and the distinction between different contraction types. However, there is no information about the spatial origin of the individual signal components. A near-infrared sensor, in contrast to this, only detects a spatially narrowly limited region below the sensor. This sensor type only indirectly allows an inference of muscle activity, as the blood quantity, and therefore the quantity of the haemoglobin, is influenced by a contraction.

The problem to be solved is therefore that a control signal proportional to the muscle contraction has to be derived, allowing a statement about a spatially narrowly limited, well definable region in the musculature with minimal interferences and without signal artefacts. The near-infrared signal with a high spatial resolution cannot be successfully used on its own to control a technical arrangement or, for example, a prosthesis. The proportionality between the muscle force exerted and the measured signal amplitude is lacking for this.

The problem disclosed is solved in that at least one double-differential myoelectric sensor and at least one near-infrared light transmitter and receiver are arranged on the skin surface, in each case, in order to detect both the muscle activity and the blood oxygen content, or the relative change in the quantity of haemoglobin, of the muscle being observed. If the blood oxygen content or the quantity of haemoglobin in the observation region changes, less light reaches the near-infrared receiver from the near-infrared transmitter. The blood oxygen content can be determined by means of the light intensity, as explained in the prior art and the contraction inferred. The penetration depth of the light into the tissue, and therefore the observation depth, is determined by the spacing apart of the NIR transmitter and NIR receiver. As different movements have characteristic time-variant patterns in the EMG and NIR signal, the movement can be inferred by measuring the two signals and thus, for example, an arm prosthesis can be controlled successfully and in a manner close to reality.

The control signals for an arm, hand or leg prosthesis, for example, are produced by fusing the sensor signals and by combining the sensors. The myoelectric signal is used here for the general recognition of the beginning of a contraction. Following this in terms of time, in a short time period, the muscle contraction is classified within a spatially limited region by means of an NIR signal. When using a plurality of NIR sensors, the reliability of the classification and moreover also muscle activities in different muscle regions are distinguished and classified. Following the classification phase, which is short in terms of time, a movement or action of the technical arrangement, for example a prosthesis, can be carried out depending on the myoelectric signal(s).

### EMBODIMENTS OF THE INVENTION

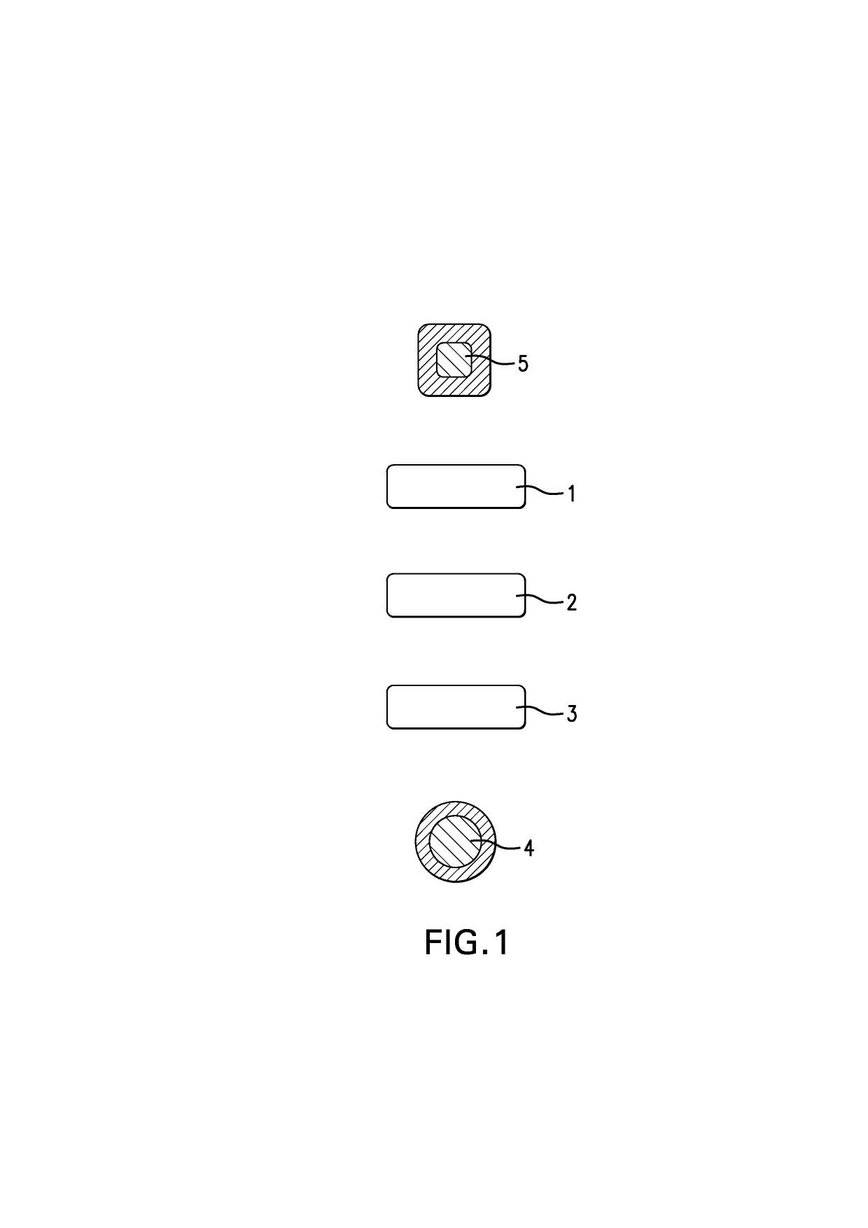

The invention consists of a plurality of electrodes and one or more near-infrared light sources and one or more optical receivers, which are arranged in a spatial relationship to one another. FIG. 1 to FIG. 5 show, by way of example, a plurality of different arrangements. In general, the electrodes are connected to one or to more double-differential EMG sensors. The spatial arrangement of the EMG sensors with respect to NIR sensors and of the NIR transmitters with respect to the NIR receivers means that precisely defined regions in the tissue are observed. The figures in each case show the support face of the sensor from the skin side.

FIG. 1 shows a centrally arranged double-differential EMG sensor, consisting of the electrode pairs (1) and (2) and (2) and (3) with which the myoelectric activity of the muscles located therebelow is detected. The EMG sensor consists of three conductive areas (1), (2) and (3), in our assemblies these are bare metal contacts with downstream instrument amplifiers of the INA 121 type from the company Texas Instruments. The near-infrared transmitter, for example an ultra-bright source (4), such as, for example, a light-emitting diode or light-emitting diode arrangement in an encapsulated housing, in our assemblies of the type L4*730/4*805/4*850-40Q96-I of the producer Epitex Incorporation, and a sensitive near-infrared receiver (5), for example a photodiode or a photo transistor with an amplifier connected downstream or integrated in a housing, in our assemblies of the OPT101 type from the producer Texas Instruments, surround, singly or in a multiple configuration, the central sensor arrangement here.

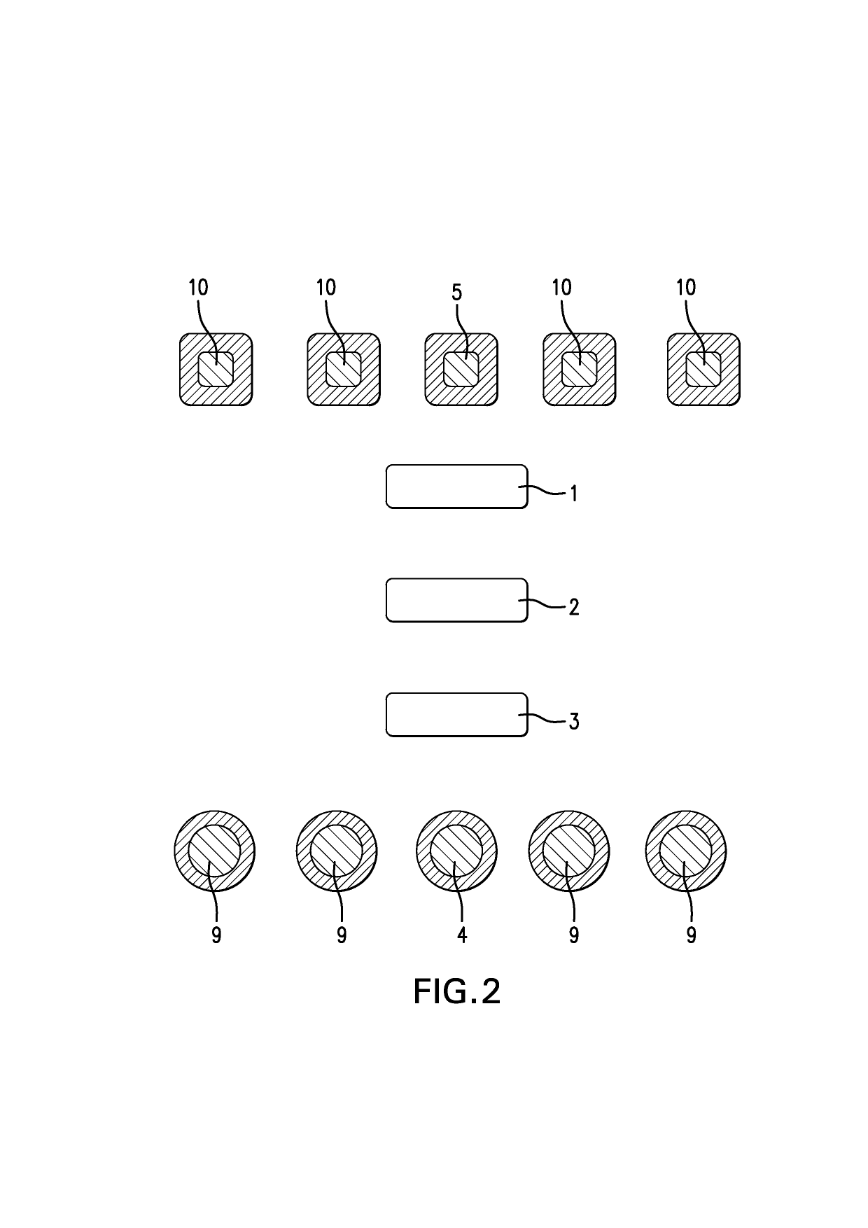

FIG. 2 shows, similarly to FIG. 1, an embodiment with a centrally arranged double-differential EMG sensor, consisting of the electrode pairs (6) and (7) and (7) and (8), with which the myoelelectrical activity of the muscles located therebelow is detected. The EMG sensor consists here of a plurality of near-infrared transmitters (9) and a plurality of near-infrared receivers (10). In this embodiment, the transmitters and receivers are arranged next to the EMG sensor such that the muscle and the tissue and the blood vessels at a depth along the muscle and slightly obliquely along the muscle can register the blood oxygen content, for example the relative quantity of the haemoglobin.

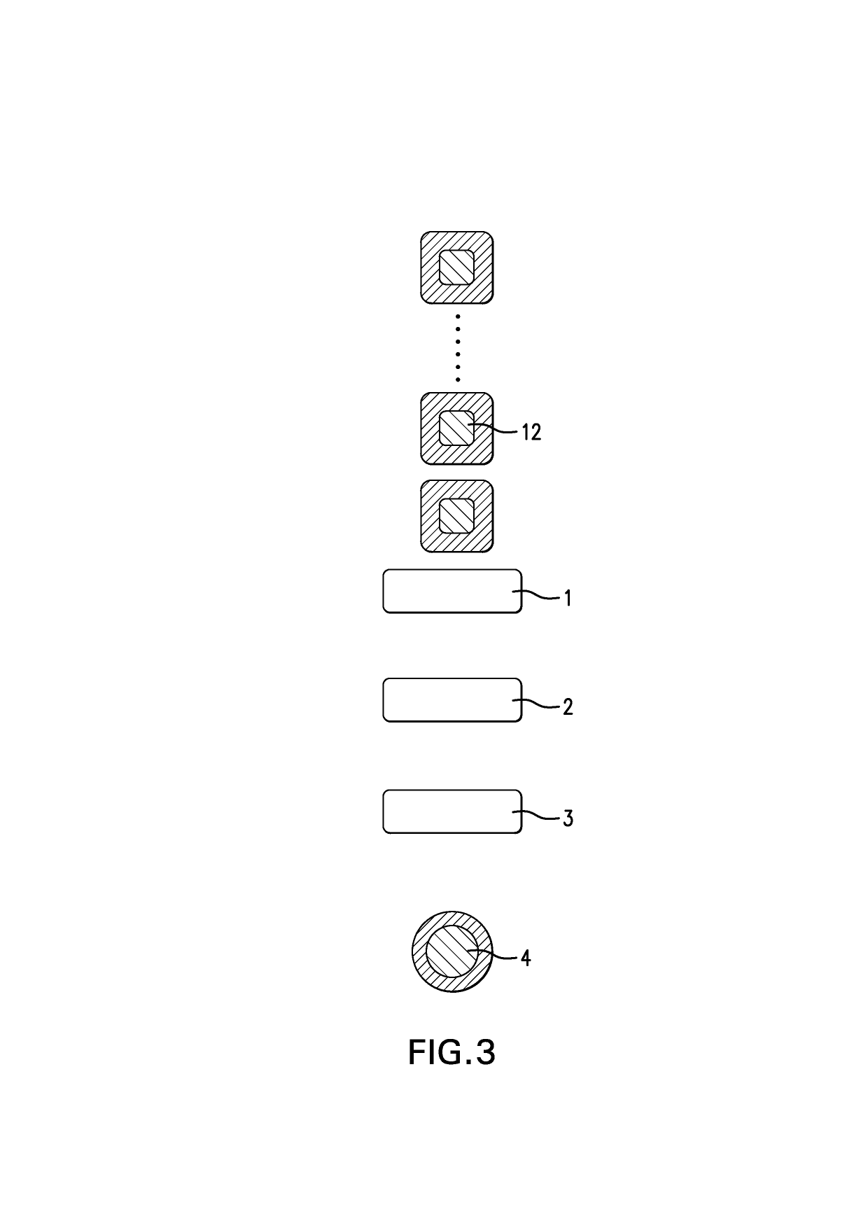

The embodiment in FIG. 3 allows the detection of the blood oxygen content, or the quantity of the haemoglobin, at different depths along or in the muscle. For this purpose, the region with an NIR transmitter (11) is illuminated and the non-absorbed remaining light is detected by the sensors (12), controlled in a clocked manner, simultaneously or consecutively, in order to measure directly under the EMG sensor at different depths.

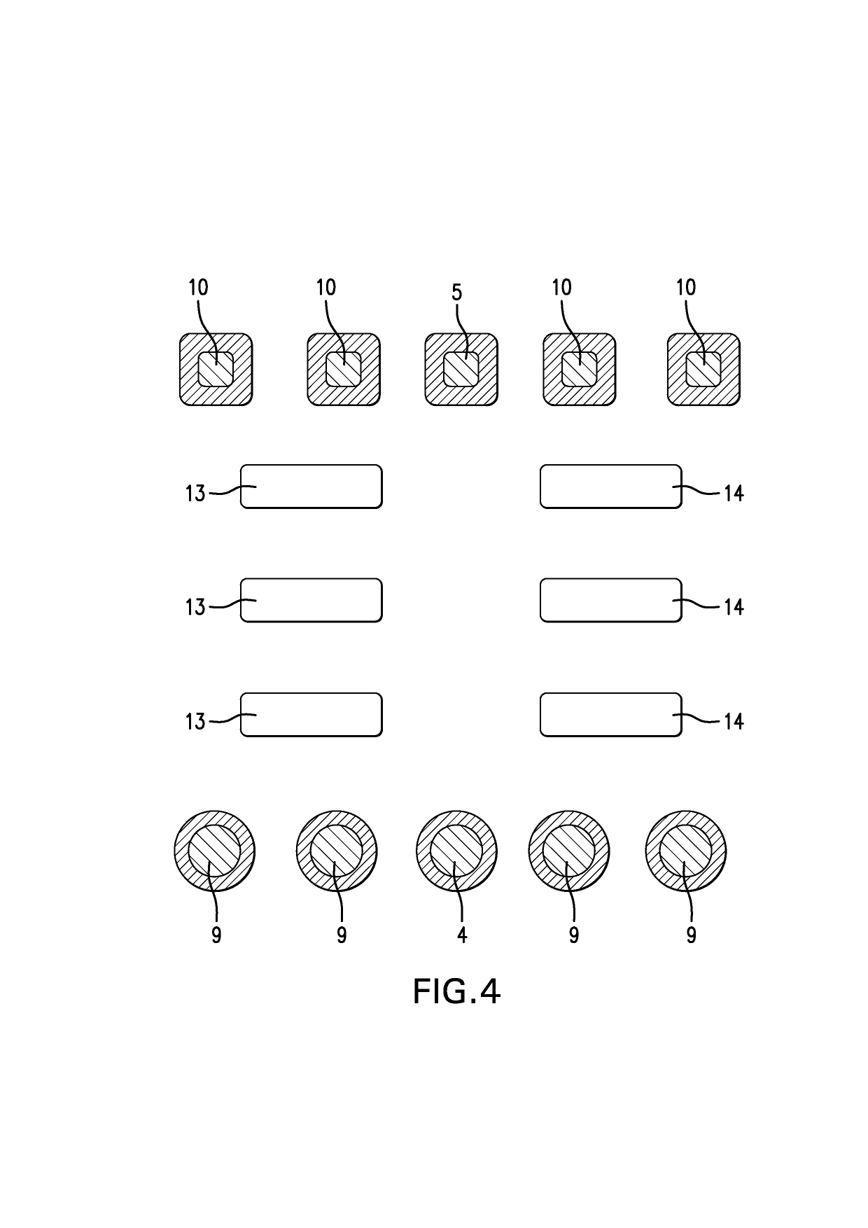

Two or more centrally arranged EMG sensors (13) and (14) allow the myoelectric signals of a large muscle, for example on the leg, or a plurality of muscles located next to one another, for example the chest region, to be detected simultaneously. The signals of the sensors (13) and (14) are used here either individually or as correlated signals, for example to reduce the noise or remove interferences or artefacts. One embodiment is shown in FIG. 4. As in FIG. 2, a plurality of near-infrared transmitters (15) and a plurality of near-infrared receivers (16) are also positioned next to the EMG sensors in this example.

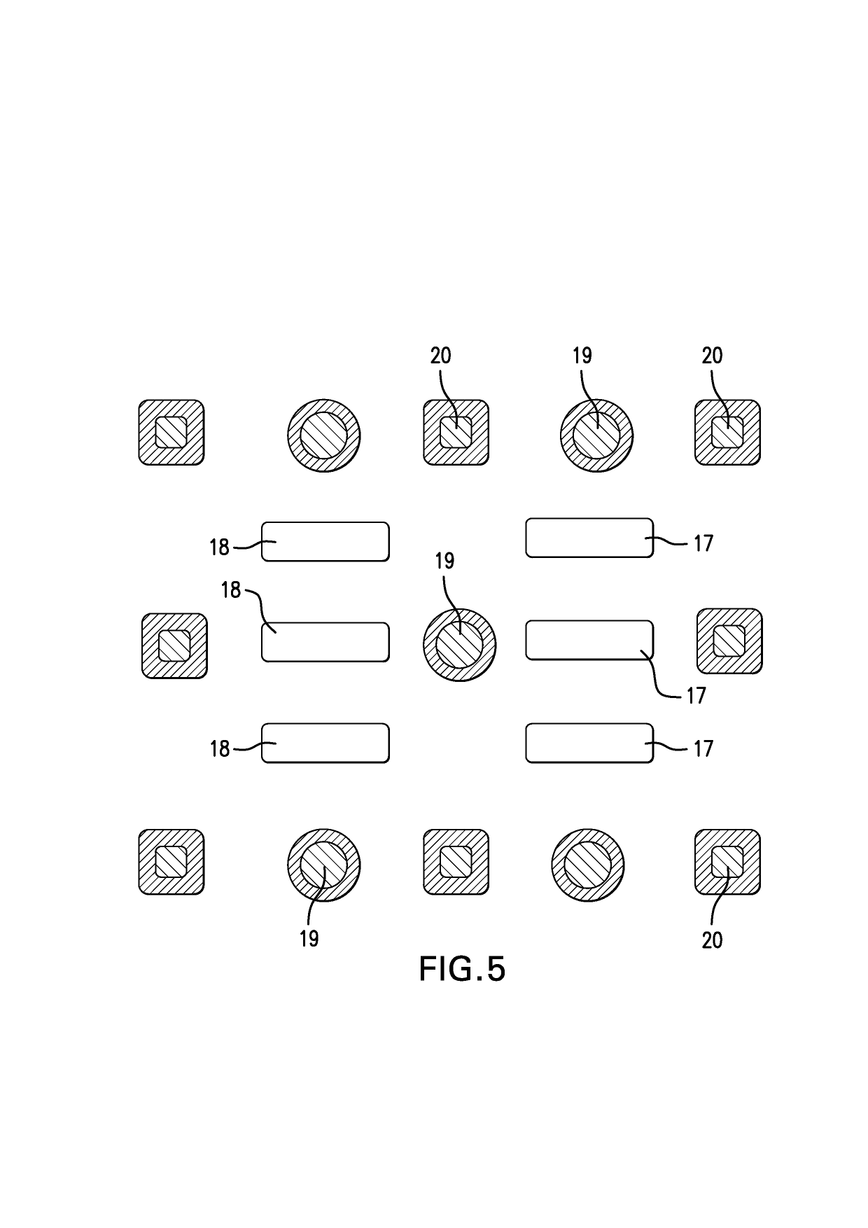

FIG. 5 shows the embodiment of a combined sensor, consisting of a plurality of EMG sensors (17) and (18) and a plurality of near-infrared transmitters (19) and (21) and a plurality of near-infrared receivers (20). This array arrangement allows the blood oxygen content, or the relative haemoglobin quantity, to be measured at different depths and regions either simultaneously or consecutively.

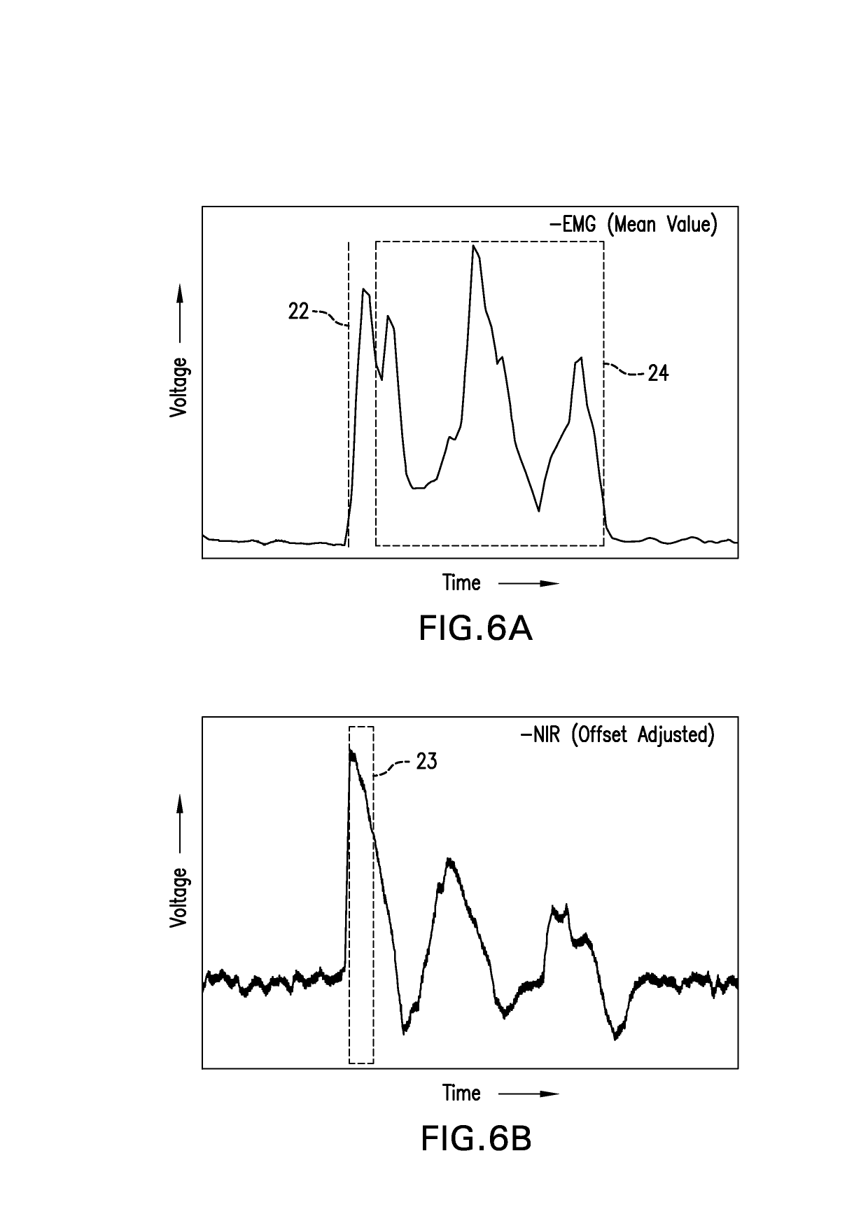

In all the arrangements, the myoelectric and the near-infrared signal are not evaluated simultaneously, but consecutively. The reason for this is the different signal properties and the information contained therein, which are based on various underlying physical effects. The mean value of myoelectric signal is approximately proportional to the muscle force exerted. However, inferences about the type of contraction exerted can hardly be drawn from a single myoelectric signal, as an overlay of signals of a plurality of muscles occurs, it not being possible to determine the origin. If, as, for example, in the forearm, a plurality of muscles are located in the surroundings of the EMG sensor, a distinction cannot be made as to which one has carried out a contraction. Owing to the fixed and spatially delimited observation region of the near-infrared sensor, different contractions can be inferred with the aid of a sensor. However, the resulting signal is not proportional to the force developed and also only supplies meaningful information at the beginning of a contraction. For this reason, the myoelectric signal is firstly used for recognition as to whether a muscle contraction is present at all. The NIR signal cannot be used for this, as it is subject to interferences due to pressure changes or changes in the surrounding light. Once a contraction has been recognised, a short time period can be used in the NIR signal to classify the type of contraction. As no inferences as to the strength of the contraction are possible by this, however, the myoelectric signal is used again following the classification in order to control a movement proportionally, in other words depending on the muscle force exerted. FIG. 6 shows a derived EMG and NIR signal for a hand movement over the same time axis. Once a predefined threshold value (22) has been exceeded, the movement is determined in the time window for the classification (23) and the intensity of the movement is determined by the course of the EMG signal amplitude in the time window (24). The movement and force control, for example of a hand prosthesis, is determined therefrom by calculation and by known control engineering algorithms.

### Literature References:

[ALTER] Alter, Ralph (1966), Bioelectric Control of Prostheses, Technical Report: Massachusetts Institute of Technology, Research Laboratory of Electronics
[AKIMA] Akima, H. (2005), Functional Imaging of Human Skeletal Muscle During Movement: Implications for Recruitment, Metabolism and Circulation. International Journal of Sport and Health Science, Vol. 3, pp. 194-207.
[ASLIN] Aslin, R. and Mehler J. (2005), Near-infrared spectroscopy for functional studies of brain activity in human infants: promise, prospects and challenges. Journal of Biomedical Optics, Vol. 10. pp. 011009.
[CHALM] Chalmers, J. and Griffiths, P. eds. (2001), Handbook of Vibrational Spectroscopy. John Wiley & Sons.
[DELSYS] De Luca, C. J. (2007), A Practicum on the Use of sEMG Signals in Movement Sciences. Delsys Inc.
[FARINA] Farina, D., Merletti, R., Indino, B., Nazzaro, M. and Pozzo, M. (2002), Surface EMG crosstalk between knee extensor muscles: Experimental and model results. Muscle & Nerve, 26, pp. 681-695.
[HERR09] Herrmann, S. and Buchenrieder, K. (2009), Effects of Muscle Fatigue on Myoelectric Signal and Features in the Time-Domain (2009). African Journal of Information and Communication Technology. Eingereicht 2009.
[HERR10A] Herrmann, S. and Buchenrieder, K. (2010), Advanced Control Schemes for Upper Limb Prostheses. Advances in Medicine and Biology, Nova Science Publishers, Vol. 15, Chapter 17.
[HERR10B] Herrmann, S. and Buchenrieder, K. (2010), Development of a Combined Myoelectric and Near-infrared Sensor for Prostheses Control (2010). Proceedings of the 7th IASTED International Conference on Biomedical Engineering BioMed 2010, pp. 181-187.
[HERR10C] Herrmann, S. and Buchenrieder, K. (2010), Fusion of Myoelectric and Near-infrared Signals for Prostheses Control. Proceedings of the 4th International Convention on Rehabilitation Engineering & Assistive Technology, Article No: 54.
[HERR11A] Herrmann, S., Attenberger, A. and Buchenrieder, K. (2011), Prostheses Control with Combined Near-infrared and Myoelectric Signals. EUROCAST 2011, Part II, LNCS 6928, pp. 602-609.
[HERR11B] Herrmann, S. (2011), Direkte and proportionate Ansteuerung einzelner Finger von Handprothesen. Dissertation, University of the Federal Armed Forces, Munich, Faculty of Information Technology, Graduation date: 25. November 2011.
[LUCA] De Luca, Carlo (2006), Electromyography. Encyclopedia of Medical Devices and Instrumentation, John Wiley Publisher, pp. 98-109
[MIURA01] Miura, H., McCully, K., Hong, L., Nioka, S. and Chance, B. (2001), Regional Difference of Muscle Oxygen Saturation and Blood Volume during Exercise Determined by Near Infrared Imaging Device. Japanese Journal of Physiology, Vol. 51, pp. 599-606.
[MIURA03] Miura, H., McCully, K. and Chance, B. (2003), Application of

Multiple NIRS imaging device to the exercising muscle metabolism. Spectroscopy, Vol. 17, pp. 549-558.
[QUARE] Quaresima, V., Colier, W., van der Sluijs, M., and Ferrari (2001), Nonuniform Quadriceps 0₂ Consumption Revealed by Near Infrared Multipoint Measurements. Biochemical and Biophysical Research Communications, Vol. 285, pp. 1034-1039.

### Captions

Abbildung=figure

### FIG. 6:

Spannung=voltage
Mittelwert=mean value
Offset bereinigt=offset adjusted
Zeit=time

1. A sensor arrangement for detecting muscle activity for the control of technical equipment, which arrangement when in use covers a region of the skin surface of a user, to provide a signal indicative of muscle activity in a limited region for subsequent processing, wherein the arrangement comprises at least one double-differential myoelectric sensor together with at least one near-infrared sensor for simultaneous or time-delayed derivation of (a) myoelectric activity and (b) the value of a parameter of the blood (for example the blood oxygen content or the relative quantity of haemoglobin) respectively, in the muscle, the muscles or the tissue under the arrangement.

2. A sensor arrangement according to claim 1, wherein the said at least one near-infrared sensor comprises a near-infrared transmitter and a plurality of near-infrared receivers, and in that an electrode of the said at least one double-differential myoelectric sensor, the near-infrared transmitter and the plurality of near-infrared receivers, are arranged linearly, in an array, in an arcuate configuration or in a circular configuration, to enable measurement of a parameter of the blood, such as the blood oxygen content or the relative quantity of haemoglobin, in different depths, layers or regions from which myoelectric signals are detected by the said electrode.

3. A sensor arrangement according to claim 1, wherein the said at least one near-infrared sensor comprises a plurality of near-infrared transmitters and a near-infrared receiver, and wherein an electrode of the said at least one double-differential myoelectric sensor the plurality of near-infrared transmitters and the near infrared receiver, are arranged linearly, in an array, in an arcuate configuration or in a circular configuration, to enable a measurement of a parameter of the blood, such as the blood oxygen content or the relative quantity of haemoglobin, in different depths, layers or regions from which myoelectric signals are detected by the said electrode.

4. A sensor arrangement according to claim 1, wherein the said at least one near-infrared sensor comprises one or more near-infrared transmitters and one or more near-infrared receivers, and wherein an electrode of the said at least one double-differential myoelectric sensor, the said one or more near-infrared transmitters and the said one or more near-infrared receivers, are arranged linearly, in an array, in an arcuate configuration or in a circular configuration, to enable a measurement of a parameter of the blood, such as the blood oxygen content or the relative quantity of haemoglobin, in different depths, layers or regions from which myoelectric signals are detected by the said electrode.

5. A sensor arrangement according to claim 1, wherein one or more electrodes of a myoelectric sensor of the arrangement, or one or more near-infrared transmitters or one or more near-infrared receivers of the near-infrared sensor are operated in pulsed mode, the pulses of which operation overlap or to not overlap as a function of time, in order to decouple signals, or to couple them, or to keep the mutual influencing of the signals low, or to exclude it, or to save energy.

6. A sensor arrangement according to claim 1, wherein signals indicative of the derived myoelectric activity and the value of parameter of the blood are not evaluated simultaneously, but consecutively, wherein
in the rest state, in which no muscle activity is present, the beginning of a contraction, as indicated by a threshold value of the signal indicative of myoelectric activity being exceeded, is awaited, and thereupon
in the movement state, in which muscle activity is present, the signal from the at least one near-infrared sensor indicative of the value of a parameter of the blood is used to classify the movement, the contraction or the body part position.

7. A sensor arrangement according to claim 6, wherein following the classification, a signal from the said at least one double-differential myoelectric sensor is used to calculate or obtain for the subsequent processing, a control signal, which is generally proportional to the muscle force exerted.

8. A method of detecting muscle activity for the control of technical equipment using a sensor arrangement as claimed in claim 1.
